(19) United States
(12) Patent Application Publication
(10) Pub. No.: US 2010/0331424 A1
(43) Pub. Date: Dec. 30, 2010

(54) Use of Axomadol for Treatment of Arthrosis Pain

(75) Inventors: Klaus Schiene, Juechen (DE); Petra Bloms-Funke, Wuerselen (DE); John Bothmer, Heerlen (NL); Claudia Lefeber, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 12/879,562

(22) Filed: Sep. 10, 2010

Related U.S. Application Data
(63) Continuation of application No. 12/118,231, filed on May 9, 2008.

(30) Foreign Application Priority Data
May 11, 2007 (DE) 10 2007 022 790.8

(51) Int. Cl. A61K 31/138; A61P 29/00; A61P 19/02

(52) U.S. Cl. 514/650

(57) ABSTRACT

The use of axomadol for the treatment of pain in arthrosis.

### CROSS REFERENCE TO RELATED APPLICATIONS

This application is a continuation of application Ser. No. 12/118,231, filed May 9, 2008 and now abandoned. Priority is claimed based on Federal Republic of Germany patent application no. DE 10 2007 022 790.8, filed May 11, 2007.

### BACKGROUND OF THE INVENTION

The invention relates to the use of axomadol for the treatment of pain in the case of arthrosis.

Arthrosis (osteoarthritis, arthritis deformans) is the most widespread joint disease in humans. It is a dynamic, but slowly progressive degenerative disease of the cartilage and other joint tissue, primarily in older individuals, with intermittent inflammation episodes. It can be distinguished from other rheumatic diseases because of the absence of inflammation parameters, the restricted mobility, short-term joint stiffness and radiological indications.

Arthrosis or wear on a joint is joint damage that starts with degeneration of the cartilage of the joint. In severe cases, it ultimately results in metaplastic processes in the adjacent bone, which destroys the surface of the joint. Therefore, the effects of the disease are pain and stiffness of the joint with restrictions in movement. The joints can become deformed and ultimately become completely ossified. Arthrosis is generally a slowly progressing process. The cartilage layer subsequently firstly thickens and the chondrocytes become metabolically more active. Changes in the subchondral trabecula lead to reduced pressure relief by the spongy bone. The regenerative tissue is more heavily stressed and as the disease progresses the balance changes towards destruction. A narrowing of the joint cavity becomes visible through radiology and osteophytes are formed at the edges. For further details, reference can be made in full, for example, to D. Hoffler et al., Therapy Recommendations of the Drug Commission of the German Medical Association, drug prescription in practice, “Degenerative Joint diseases”, 2nd edition 2001; and H. Bröll et al., CliniCum, special edition September 2001, Consensus Statement, “Arthrosis, Diagnosis & Therapy”.

In principle, all joints can be affected by arthrotic changes. However, those most affected are the knee joints (gonarthrosis) and hip joints (coxarthrosis), which carry a substantial weight load. The disease also frequently occurs in the small joints of the spine (spondylarthrosis) and also in the finger joints. According to ICD-10 arthrosis of the hip and of the knee are defined as primary cartilage diseases, which are associated with painful restrictions in movement (impact pain, weight-bearing pain) or walking disabilities. Inflammation such as synovitis can be established, but does not have to be.

Principal and early symptoms of arthrosis are pain (early triad: impact pain, fatigue pain, weight-bearing pain; late triad: continuous pain, night pain, muscle pain). They are accompanied by restrictions in movement, sensitivity to weather changes, crepitation. The causes of pain in the case of arthrosis principally result from irritations in periarticular tendon and ligament attachments, secondary inflammation, joint capsule expansion, discharge as a result of irritation, increased pressure in the subchondral bone and microfractures.

In early stages pain only occurs when bearing weight and eases again after a few minutes upon continued movement, e.g. during walking for longer periods. If inflammation additionally occurs, the typical complaints of activated arthrosis are evident: the joint is painful, it feels warm and is swollen. Mobility is restricted. The inflammation often subsides without treatment. This explains the generally intermittent course of arthrosis: phases of severe pain and restriction to movement alternate with phases of little pain and good mobility. As the attrition signs progress further one pain phase will be followed more quickly by another. Finally, the pain will remain constant.

There are many alternative non-drug and drug treatments available that are used individually or in combination:
general measures, e.g. swimming, cycling, targeted exercise, use of working aids, diet etc.;
physical therapy, e.g. heat packs, electrotherapy and movement therapy, etc.;
pharmacotherapy;
orthopaedic aids, e.g. bandages, orthotic devices, etc.; and
surgical therapy, e.g. transplantation of autologous cartilage cells, artificial joint replacement, etc.

To evaluate the success of a specific therapy, the European League Against Rheumatism (EULAR) recommends the Lequesne Index, i.e. the global evaluation by the physician and the pain assessment of the patient. Besides the assessment of swelling, reddening and pressure resistance of the joint, the FDA recommends assessment of the pain and function by means of the Western Ontario McMaster Universities Osteoarthritis Index (WOMAC) and the Lequesne Index. For drugs used to treat the symptoms of arthrosis, the Osteoarthritis Research Society recommends the scales of the WOMAC pain score as main target criterion and as secondary target criterion the movement restriction score of WOMAC or the Lequesne Index, and additionally the global evaluation by the physician and patient.

The pharmacotherapeutic spectrum of the groups of active ingredients available for therapy for arthrosis comprises
non-opioid analgesics, e.g. paracetamol;
nonsteroidal antirheumatic/antiphlogistic drugs (NSAR), e.g. acemetacin, acetylsalicylic acid, aceclofenac, diclofenac, ibuprofen, ketoprofen, mefenamic acid, tiaprofenic acid, indomethacin, lonazolac, naproxen, proglumetacin, meloxicam, piroxicam, rofecoxib, celecoxib;
opioid analgesics, e.g. dihydrocodeine, tramadol, tilidine-naloxone, morphine, buprenorphine, oxycodone, fentanyl and hydromorphone;
percutaneously administered antiphlogistic drugs and hyperemics;
glucocorticosteroid crystal suspensions for intra-articular injections; and
further active agents for oral or intra-articular injections, e.g. glucosamine, ademetionine, oxaceprol, hyaluronic acid, etc.

Opioid analgesics are not routinely part of the repertoire in the drug treatment of arthrosis, but are unavoidable in certain situations. However, conventional opioid analgesics exhibit significant side-effects in some cases, in particular constipation, nausea, vomiting, headaches, sedation, tiredness, respiratory depression, allergies and occasionally drop in blood pressure. These side-effects make any long-term therapy of chronic pain in arthrosis difficult. Therefore, treatment with conventional opioid analgesics is generally only indicated after all other therapeutic possibilities have been exhausted, e.g. in patients who cannot be operated on, but suffer from extreme rest pain that does not respond to other analgesically active substances.

There is a need for alternative pharmacotherapeutic methods of treatment for arthrosis, which are distinguished by an effective alleviation of pain and an improved side-effect profile.

### SUMMARY OF THE INVENTION

Therefore, it is an object of the invention to find compounds that are effective in the alleviation of pain in arthrosis and have advantages over conventional analgesics.

These and other objects are achieved by the invention as described and claimed hereinafter.

The invention relates in particular to the use of axomadol for the treatment of pain in the case of arthrosis.

It has surprisingly been found that axomadol combines an excellent efficacy in the treatment of pain in arthrosis and a reduced side-effect spectrum. Moreover, it has been found that in the chronic inflammation pain model, axomadol shows a better analgesic efficacy compared to conventional analgesics such as morphine, oxycodone and tramadol, for example.

Axomadol, i.e. (1RS,3RS,6RS)-6-dimethylaminomethyl-1-(3-methoxyphenyl)-cyclohexane-1,3-diol, is a synthetic, centrally active analgesic, which is effective in the treatment of moderate to severe, acute or chronic pain. Axomadol can be used in the form of its free base or as a salt or solvate.

U.S. Pat. No. 5,733,936 (=EP 753,506) describes the synthesis of axomadol and experiments on its analgesic efficacy in the tail-flick test in mice. The stereoselective synthesis of axomadol by means of enzymatic racemate resolution of the corresponding ester precursor is known from U.S. Pat. No. 7,168,937 (=WO 01/57232). US application no. 2004/029878 (=WO 02/43714) discloses axomadol for the treatment of increased need to urinate or urinary incontinence. US application no. 2004/242617 (=WO 03/024444) describes that an active agent combination containing axomadol and a muscarine antagonist can also be used to treat urinary incontinence. US application no. 2005/176790 (=WO 02/067916) describes the solution behavior of axomadol hydrochloride salt and the solubility of axomadol saccharinate in water. US application no. 2006/121113 (=WO 2005/009329) discloses the pharmaceutical formulations of axomadol with delayed active agent release.

As used in this application, the term “axomadol” means (1RS,3RS,6RS)-6-dimethyl-aminomethyl-1-(3-methoxyphenyl)-cyclohexane-1,3-diol, its pharmaceutically compatible salts and/or solvates.

Suitable pharmaceutically compatible salts include salts of inorganic and/or organic acids such as e.g. acetic acid, 2,2-dichloroacetic acid, acylated amino acids, preferably acetylated amino acids such as e.g. N-acetylalanine, N-acetylcysteine, N-acetylglycine, N-acetylisoleucine, N-acetylleucine, N-acetylmethionine, N-acetylphenylalanine, N-acetylproline, N-acetylserine, N-acetylthreonine, N-acetyltyrosine, N-acetylvaline, adipic acid, alginic acid, ascorbic acid, L-aspartic acid, benzenesulfonic acid, benzoic acid, 4-acetamidobenzoic acid, (+)-camphoric acid, (−)-camphoric acid, (+)-camphor sulfonic acid, (−)-camphor sulfonic acid, (+)-camphor-10-sulfonic acid, (−)-camphor-10-sulfonic acid, (±)-camphor-10-sulfonic acid, capric acid, caproic acid, caprylic acid, cation exchange resins, cinnamic acid, citric acid, cyclohexyl sulfamic acid, sulfuric acid monododecyl ester, ethane-1,2-sulfonic acid, ethanesulfonic acid, 2-hydroxyethanesulfonic acid, formic acid, fumaric acid, mucic acid (galactosaccharic acid), gentisic acid, glucose monocarboxylic acid (glucoheptonic acid), d-gluconic acid, d-glucuronic acid, L-glutamic acid, α-oxoglutaric acid (α-ketoglutaric acid), hydroxyacetic acid (glycollic acid), hippuric acid (N-benzoylglycine), hydrogen bromide, hydrogen chloride, (+)-L-lactic acid, (±)-DL-lactic acid, lactobionic acid (4-O-β-D-galactopyranosyl-D-gluconic acid), maleic acid, (−)-L-malic acid, malonic acid, (±)-DL-mandelic acid, methanesulfonic acid, naphthalene-2-sulfonic acid, naphthalene-2,5-disulfonic acid, 1-hydroxy-2-naphthalene-carboxylic acid, nicotinic acid, nitric acid, oleic acid, orotic acid (uracil-6-carboxylic acid), oxalic acid, palmitic acid, pamoa acid (embonic acid), phosphoric acid, L-pyroglutamic acid, salicylic acid, acetylsalicylic acid, 4-aminosalicylic acid, sebacic acid, stearic acid, succinic acid, sulfuric acid, tannic acid, (+)-L-tartaric acid, (±)-DL-tartaric acid, thiocyanic acid, p-toluenesulfonic acid and undecylenic acid. Preferred salts are hydrochloride, saccharinate, dihydrogen phosphate, hydrogen phosphate and phosphate. Axomadol can also be present as a mixture of salts of the above-mentioned organic and inorganic acids in any desired ratio.

In a preferred embodiment, the medication is a solid drug form. The medication is preferably manufactured for oral administration. However, other forms of administration are also possible, e.g. for buccal, sublingual, transmucosal, rectal, intralumb al, intraperitone al, transdermal, intravenous, intramuscular, intragluteal, intracutaneous and subcutaneous administration.

Depending on the configuration, the medication preferably contains suitable additives and/or adjuvants. Suitable additives and/or adjuvants in the sense of the invention include all substances known to persons skilled in the art for use in the preparation of galenic formulations. The choice of these adjuvants and also the quantities to be used are dependent on how the medication is to be administered, i.e. orally, intravenously, intraperitoneally, intradermally, intramuscularly, intranasally, buccally or locally.

Preparations suitable for oral administration include those in the form of tablets, chewable tablets, lozenges, capsules, granules, drops, liquids or syrups, and those suitable for parenteral, topical and inhalatory administration are solutions, suspensions, easily reconstituted dry preparations and sprays. A further possibility is suppositories for rectal administration. Examples of suitable percutaneous forms of administration include administration in a depot in dissolved form, a patch or a plaster, possibly with the addition of agents promoting skin penetration.

Examples of adjuvants and additives for oral forms of administration include disintegrants, lubricants, binders, fillers, mold release agents, possibly solvents, flavorings, sugar, in particular carriers, diluents, coloring agents, antioxidants etc.

Waxes or fatty acid esters, amongst others, can be used for suppositories and carrier substances, preservatives, suspension aids etc. can be used for parenteral forms of application.

Useful adjuvants may include, for example: water, ethanol, 2-propanol, glycerine, ethylene glycol, propylene glycol, polyethylene glycol, polypropylene glycol, glucose, fructose, lactose, saccharose, dextrose, molasses, starch, modified starch, gelatine, sorbitol, inositol, mannitol, microcrystalline cellulose, methyl cellulose, carboxymethyl-cellulose, cellulose acetate, shellac, cetyl alcohol, polyvinylpyrrolidone, paraffins, waxes, natural and synthetic rubbers, acacia gum, alginates, dextran, saturated and unsaturated fatty acids, stearic acid, magnesium stearate, zinc stearate, glyceryl stearate, sodium lauryl sulfate, edible oils, sesame oil, coconut oil, peanut oil, soybean oil, lecithin, sodium lactate, polyoxyethylene and propylene fatty acid esters, sorbitane fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talc, kaolin, pectin, crospovidon, agar and bentonite.

The production of these medicaments and pharmaceutical compositions is carried out using means, devices, methods and processes that are well known in the art of pharmaceutical technology, as described, for example, in “Remington's Pharmaceutical Sciences”, A. R. Gennaro, 17th ed., Mack Publishing Company, Easton, Pa. (1985), in particular in part 8, chapters 76 to 93.

Thus, for example, for a solid formulation such as a tablet, the active substance of the drug can be granulated with a pharmaceutical carrier substance, e.g. conventional tablet constituents such as cornstarch, lactose, saccharose, sorbitol, talc, magnesium stearate, dicalcium phosphate or pharmaceutically acceptable rubbers, and pharmaceutical diluents such as water, for example, in order to form a solid composition that contains the active substance in a homogenous dispersion. Homogenous dispersion is understood here to mean that the active substance is uniformly dispersed throughout the composition, so that this can be readily divided into identically effective standard dose forms such as tablets, capsules, lozenges. The solid composition is then divided into standard dose forms. The tablets or pills can also be coated or otherwise compounded to prepare a slow release dose form. Suitable coating agents include polymeric acids and mixtures of polymeric acids with materials such as shellac, cetyl alcohol and/or cellulose acetate, for example.

The quantities of axomadol to be administered to patients vary depending on the weight of the patient, the type of application and the severity of the illness. In a preferred embodiment, the medication contains axomadol in a quantity of 10 to 2000 mg, more preferred 15 to 1000 mg, and still more preferred 20 to 500 mg, based on the free base.

Axomadol can be released slowly from preparations that can be administered orally, rectally or percutaneously. The medication is preferably manufactured for administration twice daily (bid), or three times daily, the twice daily administration (bid) being particularly preferred. A slow release of axomadol can be achieved, for example, by retardation using a matrix, a coating or osmotically active release systems (cf. US 2006/121113, for example).

In a preferred embodiment:
the medication is manufactured for oral administration; and/or
the medication is a solid and/or compressed and/or film-coated drug form; and/or
the medication releases axomadol slowly from a matrix; and/or
the medication contains axomadol in a quantity of 0.001 to 99.999% by wt., more preferably 0.1 to 99.9% by wt., still more preferably 1.0 to 99.0% by wt., even more preferably 2.5 to 80% by wt., most preferably 5.0 to 50% by wt. and in particular 7.5 to 40% by wt., based on the total weight of the medication; and/or
the medication contains a pharmaceutically compatible carrier and/or pharmaceutically compatible adjuvants; and/or
the medication has a total mass in the range of 25 to 2000 mg, more preferred 50 to 1800 mg, still more preferred 60 to 1600 mg, more preferred 70 to 1400 mg, most preferred 80 to 1200 mg and in particular 100 to 1000 mg; and/or
the medication is selected from the group consisting of tablets, capsules, pellets and granules.

The medication can be provided as a simple tablet or as a coated tablet (e.g. as film-coated tablet or lozenge). The tablets are usually round and biconvex, but oblong forms are also possible. Granules, spheres, pellets or microcapsules, which are contained in sachets or capsules or are compressed to form disintegrating tablets, are also possible.

Medications containing at least 0.001 to 99.999% by wt. axomadol, in particular low effective doses, are preferred to avoid side-effects. The medication preferably contains 0.01% by wt. to 99.99% by wt. axomadol, more preferred 0.1 to 90% by wt., still more preferred 0.5 to 80% by wt., most preferred 1.0 to 50% by wt. and in particular 5.0 to 20% by wt.

It is particularly preferred if the medication is in a form for oral administration that is configured for twice daily application and contains axomadol in a quantity of 10 to 2000 mg based on the free base.

Axomadol exhibits a pronounced antihyperalgesic efficiency, which has been determined in the Complete Freund's Adjuvant (CFA) animal model.

According to the invention, axomadol is used for the treatment of pain in the case of arthrosis. The arthrosis is preferably selected from the group comprising gonarthrosis, coxarthrosis and spondylarthrosis.

The painful arthrosis is preferably an arthrosis in accordance with the ICD-10 (International Classification of Diseases and Related Health Problems, WHO edition, preferably 2007). The arthrosis is preferably selected from polyarthrosis [M15], coxarthrosis [M16], gonarthrosis [M17], arthrosis of the first carpometacarpal joint [M18], other arthrosis [M19] and arthrosis of the spine [M47]. The indications given in brackets relate to the nomenclature used in the ICD-10.

If the arthrosis in question is polyarthrosis [M15], then this is preferably selected from the group comprising primary generalised (osteo)arthrosis [M15.0], Heberden's nodes (with arthropathy) [M15.1], Bouchard's nodes (with arthropathy) [M15.2], secondary multiple arthrosis (posttraumatic polyarthrosis) [M15.3], erosive (osteo)arthrosis [M15.4], other polyarthrosis [M15.8] and polyarthrosis, not further specified (generalized (osteo) arthrosis, unspecified) [M15.9].

If the arthrosis in question is coxarthrosis [M16], then this is preferably selected from the group comprising bilateral primary coxarthrosis [M16.0], other primary coxarthrosis (unilateral or unspecified) [M16.1], bilateral coxarthrosis resulting from dysplasia [M16.2], other dysplastic coxarthrosis (unilateral or unspecified) [M16.3], bilateral posttraumatic coxarthrosis [M16.4], other posttraumatic coxarthrosis [M16.5] (unilateral or unspecified), other bilateral secondary coxarthrosis [M16.6], other secondary coxarthrosis (unilateral or unspecified) [M16.7] and coxarthrosis, not further specified [M16.9].

If the arthrosis in question is gonarthrosis [M17], then this is preferably selected from the group comprising bilateral primary gonarthrosis [M17.0], other primary gonarthrosis (unilateral or unspecified) [M17.1], bilateral posttraumatic gonarthrosis [M17.2], other posttraumatic gonarthrosis [M17.3] (unilateral or unspecified), other bilateral secondary gonarthrosis [M17.4], other secondary gonarthrosis (unilateral or unspecified) [M17.5] and gonarthrosis, not further specified [M17.9].

If the arthrosis in question is arthrosis of the first carpometacarpal joint [M18], then this is preferably selected from the group comprising bilateral primary arthrosis of the first carpometacarpal joint [M18.0], other primary arthrosis of the first carpometacarpal joint (unilateral or unspecified) [M18.1], bilateral posttraumatic arthrosis of the first carpometacarpal joint [M18.2], other posttraumatic arthrosis of the first carpometacarpal joint [M18.3] (unilateral or unspecified), other bilateral secondary arthrosis of the first carpometacarpal joint [M18.4], other secondary arthrosis of the first carpometacarpal joint (unilateral or unspecified) [M18.5], and arthrosis of the first carpometacarpal joint, not further specified [M18.9].

If the arthrosis in question is other arthrosis [M19], then this is preferably selected from the group comprising primary arthrosis of other joints (primary arthrosis, unspecified) [M19.0], posttraumatic arthrosis of other joints (posttraumatic arthrosis, unspecified) [M19.1], other secondary arthrosis (secondary arthrosis, unspecified) [M19.2], other further specified arthrosis [M19.8], and arthrosis, not further specified [M19.9].

Axomadol is preferably used to treat moderate to severe pain. In preferred embodiments, the pain may be selected from the group consisting of impact pain, weight-bearing pain, fatigue pain, periarticular pain caused by pressure, radiating pain (e.g. knee pain in the case of existing coxarthrosis), rest pain after remaining in the same position for a long period, continuous pain, spontaneous pain, pain on movement, night pain, muscle pain, pain in extremities and bone pain as spontaneous and rest pain. The pain is preferably hyperalgesia or allodynia. Hyperalgesia is preferably induced thermally or mechanically.

Even if the medications according to the invention only exhibit slight side-effects, it can be advantageous, for example, in order to avoid specific forms of dependency, to also use morphine antagonists, in particular naloxone, naltrexone and/or levallorphan, besides axomadol.

The invention additionally relates to a method for treating pain in the case of arthrosis, in which axomadol is administered to a patient in a pharmaceutically effective quantity.

The following examples are intended to illustrate the invention in further detail, but should not be interpreted as restrictive.

### EXAMPLES

### 1. Clinical Studies

Two studies with a respective treatment period of 4 weeks were conducted to determine the efficacy and safety of axomadol in patients with moderate to severe chronic pain as a result of osteoarthritis (arthrosis, OA) of functional class I-III. Both studies had a randomised, multicentric, double blind, double dummy, placebo- and actively controlled parallel group configuration.

Patients who were treated with axomadol exhibited a clinically significant decrease in pain intensity after 4 weeks.

In both studies over the entire study period, undesirable effects occurred more frequently in the patient groups treated with active substance than in the patient groups, to which the placebo was administered. These undesirable effects are generally typical for centrally active analgesics.

### Study A:

In this study, patients with OA of the hip or knee were divided into 5 groups. Different daily doses of axomadol were administered to the patients of 3 of these groups (44, 66 and 110 mg each twice daily based on the free base), one group was given tramadol (100 mg twice daily) and one group was treated with a placebo twice daily.

At each assessment time, a decrease in the pain intensity compared to the baseline pain was observed in all patient groups. In the full analysis set on day 29 a clinically relevant decrease in pain intensity was observed in the patient groups treated with axomadol. This improvement was not observed in the tramadol and placebo group.

A clinically relevant decrease in pain intensity in the per protocol set could be demonstrated dependent on dosage (higher efficacy at the higher dose) and the results of the patient group that was given 110 mg axomadol twice daily were statistically significant (p<0.05) compared to the placebo group.

The instance of the most frequent undesirable effects was higher in the patient group that received 110 mg axomadol hydrochloride twice daily than in every other treatment group. The most frequent undesirable effects were nausea, constipation, excessive sweating, dizziness, vomiting, headache, dry mouth and drowsiness.

### Study B:

In this study, patients with OA of the knee were divided into 4 groups. Two of the patient groups received different doses of axomadol hydrochloride: 100 mg and 150 mg based on the free base, twice daily in each case after a titration period of 2 weeks, in which the dose of axomadol was increased on a weekly basis. A further patient group was given oxycodone CR (20 mg twice daily) after a titration period, in which oxycodone CR was increased from 10 mg to 20 mg twice daily. A further group was given a placebo twice daily.

Analysis of the average pain intensity of the last 24 hours on day 29 for the patient group given 100 mg axomadol twice daily showed a statistically significant difference (p=0.0190) compared to the placebo in the full analysis set.

In the per protocol set, both axomadol groups (p=0.0068 for the 100 axomadol group and p=0.0079 for the 150 mg axomadol group) as well as the oxycodone group (p=0.0154) showed a statistically significant difference for the primary endpoint compared to the placebo group. Different secondary endpoints confirmed these results for the full analysis set and also for the per protocol set.

In study B more undesirable effects arose in the three active groups than in the placebo group. These undesirable effects were generally slight to moderate in most cases for the axomadol groups. However, the frequency of undesirable side-effects, which caused individual patients to discontinue the study, was twice as high in the oxycodone group (31.5% of patients) as in the two axomadol groups (16.3% in the 100 mg group and 17.7% in the 150 mg group). The most frequent undesirable effects were constipation, nausea, vomiting and dry mouth.

### 2. Antihyperalgesic Effect in the Chronic Inflammation Pain Model

The test to determine the antihyperalgesic effect of axomadol in chronic inflammation pain was conducted on rats in the Complete Freund's Adjuvant animal model (CFA).

A model for chronic inflammation represents the monoarthritis triggered by the Complete Freund's Adjuvant (CFA). By injecting a small quantity of CFA (100 μg M. tuberculosis) into the back paw a local inflammatory reaction restricted in time to 2-4 weeks occurred. The hyperalgesia occurring in parallel (hence the name CFA-hyperalgesia (CFA-HA) used hereafter) to mechanical or thermal stimuli is restricted to the inflamed paw.

Sprague Dawley rats from a commercial breeder (Janvier, Belgium) having a weight of 140-160 g were used as test animals. The CFA-HA was induced in rats by subplantar injection of CFA (100 μl of the 1 mg/ml mycobacteria (heat-killed M. tuberculosis)/oil suspension (IFA; Difco)) into the back paw (ipsilaterally). The injection day was defined as day 0 (d 0).

Hyperalgesia to a mechanical tactile stimulus was detected by means of an electronic von Frey measuring instrument (Somedic Electronic von Frey System, Somedic Sales AB, Horby, Sweden). The paw was stimulated by subplantar application. To quantify the sensitivity of both the ipsi- and the contralateral paw to the mechanical stimulus, the paw pull-away threshold was given in grams of applied pressure. The median was formed from the four measured values per paw. The pull-away threshold of the ipsi- and contralateral paw was determined on day 1 after CFA injection before (=initial value) and at different times (15, 20 and 60 minutes) after substance dose (measured value). The efficacy of a substance was calculated as % inhibition of hyperalgesia as follows:

% inhibition of HA=(1−HA measured value/HA initial value)×100

HA initial value=pull away threshold contralateral−pull away threshold ipsilateral before substance dose

HA measured value=pull away threshold contralateral−pull away threshold ipsilateral after substance dose

In total, 10 rats were used in each test animal group. The mean±SEM was calculated from the medians of the individual animals. The significance calculation was conducted using the two-factor analysis of the variance (ANOVA) for repeated measurements. In the case of a significant treatment effect, a comparison in pairs was conducted at different measurement times by a Fisher's significance test, followed by a post hoc Dunnett test. The results were assessed as statistically significant at p<0.05.

In order to determine the maximum efficacy, the substance was administered intravenously (IV) up to the maximum possible dose. The highest possible dose was defined as the dose that still exhibited no side-effects influencing the measurements and no severe antinociceptive effect on the untreated paw and a further increase in dose cased these effects. The maximum efficacy was determined based on the maximum achievable inhibition of hyperalgesia that could be reached in a dose range, which 1. did not induce any overlap with antinociceptive effects on the contralateral paw and/or 2. did not induce any side-effects to such an extent as to influence interpretation of the measured values.

### Results:

Axomadol significantly reduces CFA-induced hyperalgesia. A maximum antihyperalgesic effect of 40% was reached after intravenous application of 10 mg/kg axomadol-HCl. Higher doses led to a decrease in the antihyperalgesic effect to 9% and also an overlap with an additional antinociceptive effect.

In comparison to axomadol-HCl, a clearly lower maximum effect was achieved after application of the centrally active analgesics morphine, oxycodone and tramadol (see Table) (in each case <30% inhibition of hyperalgesia).

The results of the tests with axomadol-HCl as well as further centrally active analgesics are collated in the following Table:

| | | Dose | | |
|---|---|---|---|---|
| | | [mg/kg] | % Inhibition | |
| | Substance | IV | of HA | Remarks |
| Invention | Axomadol-HCl | 2.15 | 16 | Max. effect = 40% at 10 mg/kg. |
| | | 4.64 | 31 | From 14.7 mg/kg: overlap with antinociceptive |
| | | 10.0 | 40 | effect. |
| | | 14.7 | 9 | |
| Comparison | Morphine | 0.46 | 8 | Max. effect = 27% at 2.15 mg/kg. |
| | | 1.00 | 20 | From 4.64 mg/kg: overlap with antinociceptive |
| | | 2.15 | 27 | effect as well as occurrence of side-effects that |
| | | 4.64 | 18 | impair measurement. |
| | Oxycodone | 0.215 | 18 | Max. effect = 26% at 0.681 mg/kg. |
| | | 0.464 | 12 | From 0.681 mg/kg: overlap with antinociceptive |
| | | 0.681 | 26 | effect as well as occurrence of side-effects that |
| | | | | impair measurement. |
| | Tramadol | 2.15 | 5 | Max. effect = 29% at 4.64 mg/kg. |
| | | 4.64 | 29 | From 4.64 mg/kg: overlap with antinociceptive |
| | | 6.81 | 28 | effect as well as occurrence of side-effects that |
| | | | | impair measurement. |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of treating or inhibiting arthrosis pain in a subject, said method comprising administering to said subject a pharmacologically effective amount of axomadol.

2. A method according to claim 1, wherein the axomadol is administered in a solid drug form.

3. A method according to claim 1, wherein the axomadol is administered orally.

4. A method according to claim 1, wherein the axomadol is administered twice daily.

5. A method according to claim 1, wherein the axomadol is administered in a dose of from 10 to 2000 mg, relative to weight of the free base.

6. A method according to claim 1, wherein the axomadol is administered in the form of a hydrochloride salt.

7. A method according to claim 1, wherein said subject is a subject suffering from an arthrosis selected from the group consisting of gonarthrosis, coxarthrosis and spondylarthrosis.

8. A method according to claim 1, wherein said subject is suffering from moderate to severe arthrosis pain.

9. A method according to claim 1, wherein said pain is selected from the group consisting of impact pain, weight-bearing pain, fatigue pain, periarticular pain caused by pressure, radiating pain, rest pain after remaining in the same position for a long period, continuous pain, spontaneous pain, pain on movement, night pain, muscle pain, pain in extremities and bone pain as spontaneous and rest pain.
